(19) United States
(12) Patent Application Publication
(10) Pub. No.: US 2025/0002953 A1
(43) Pub. Date: Jan. 2, 2025

(54) HIGH EFFICIENCY PRODUCTION OF CANNABIGEROLIC ACID AND CANNABIDIOLIC ACID

(71) Applicant: Amyris, Inc., Emeryville, CA (US)

(72) Inventors: William E. Draper, Emeryville, CA (US); John E. Hung, Emeryville, CA (US); Alex McGill, Emeryville, CA (US); Victor Holmes, Emeryville, CA (US)

(21) Appl. No.: 18/578,903

(22) PCT Filed: Jul. 11, 2022
(86) PCT No.: PCT/US2022/073587

§ 371 Date: Jan. 12, 2024

Related U.S. Application Data
(60) Provisional application No. 63/221,175, filed on Jul. 13, 2021.

(51) Int. Cl. C12P 17/06; C12N 9/00; C12N 9/02; C12N 9/10; C12N 9/88; C12P 7/42

(52) U.S. Cl. CPC C12P 17/06; C12N 9/0008; C12N 9/1029; C12N 9/88; C12N 9/93; C12P 7/42; C12Y 121/03008; C12Y 203/01206; C12Y 404/01026; C12Y 602/01; C12Y 602/01002

(57) ABSTRACT

The present disclosure features compositions and methods for producing one or more cannabinoids, such as cannabidiolic acid (CBDa) or cannabidiol (CBD), in a host cell, such as a yeast cell, that is genetically modified to express the enzymes of a cannabinoid biosynthetic pathway. Using the compositions and methods of the present invention, the host cell may be genetically modified to express one or more enzymes of a cannabinoid biosynthetic pathway, such as CBDaS. The host cell may be cultured in the medium in the presence of a first cannabinoid, for example CBGa, and incubated for a time sufficient to allow for bioconversion of the first cannabinoid to a second cannabinoid, for example CBDa, by the host cell.

### BACKGROUND OF THE INVENTION

Cannabinoids are a group of structurally related molecules defined by their ability to interact with a distinct class of receptors (cannabinoid receptors). Both naturally occurring and synthetic cannabinoids are known. Naturally occurring cannabinoids are produced primarily by the Cannabis family of plants and include cannabigerol (CBG), cannabichromene (CBC), cannabidiol (CBD), cannabinol (CBN), cannabinodiol (CBDL), cannabicyclol (CBL), cannabielsoin (CBE), cannabitriol (CBT), tetrahydrocannabinol (THC), and tetrahydrocannabinolic acid (THCa). An expanding set of synthetic variants of cannabinoids have been designed to mimic the effects of the naturally occurring molecules.

Cannabinoids may be used to improve various aspects of human health. However, producing cannabinoids in preparative amounts and in high yield has been challenging. There remains a need for compositions and methods capable of preparing cannabinoids with high efficiency and chemical selectivity.

### SUMMARY OF THE INVENTION

Provided herein are compositions and methods for the improved production of a cannabinoid, such as cannabidiolic acid (CBDa), in a host cell, such as a yeast cell. For example, using the compositions and methods described herein, a host cell may be modified to express one or more enzymes of a cannabinoid biosynthetic pathway, such as an acyl-activating enzyme (AAE), a tetraketide synthase (TKS), a cannabigerolic acid synthase (CBGaS), a geranyl pyrophosphate (GPP) synthase, and/or a CBDa synthase (CBDaS). The host cell may then be cultured in a medium, for example, in the presence of an agent that regulates expression of the one or more enzymes. The host cell may further be cultured in the medium in the presence of a first cannabinoid, for example CBGa, and incubated for a time sufficient to allow for bioconversion of the first cannabinoid to a second cannabinoid, for example CBDa, by the host cell. The second cannabinoid may then be separated from the host cell or from the medium. Alternatively, a first host cell may be cultured in a medium and incubated for a time sufficient to allow for production of a first cannabinoid, for example CBGa. A second host cell may be cultured in the medium containing the first host cell and incubated for a time sufficient to allow for bioconversion of the first cannabinoid to a second cannabinoid, for example CBDa. The second cannabinoid may then be separated from the host cell or from the medium.

In one aspect, the invention provides for a genetically modified host cell capable of producing CBGa or CBG, wherein the genetically modified host cell contains one or more heterologous nucleic acids that each, independently, encodes an acetyl-CoA thiolase, an HMG-CoA synthase, an HMG-CoA reductase, a mevalonate kinase, an IPP:DMAPP isomerase, an acyl-activating enzyme comprising the amino acid sequence of SEQ ID NO: 1, a tetraketide synthase comprising the amino acid sequence of SEQ ID NO: 2, an olivetolic acid cyclase comprising the amino acid sequence of SEQ ID NO: 3, a cannabigerolic acid synthase (CBGaS) comprising the amino acid sequence of SEQ ID NO: 4, and a geranyl pyrophosphate synthase comprising the amino acid sequence of SEQ ID NO: 5.

In one embodiment, the genetically modified host cell contains one or more of: one or more heterologous nucleic acids that each, independently, encodes a pyruvate decarboxylase comprising the amino acid sequence of SEQ ID NO: 6; modified expression of an aldehyde dehydrogenase comprising the amino acid sequence of SEQ ID NO: 7; or modified expression of an acetyl-coenzyme A synthetase comprising the amino acid sequence of SEQ ID NO: 8.

In one embodiment, the genetically modified host cell is a yeast cell or a yeast strain. In one embodiment, the yeast cell or the yeast strain is Saccharomyces cerevisiae.

In another aspect, the invention provides for a method for producing CBGa or CBG, involving: culturing the genetically modified host cell of the invention in a medium with a carbon source under conditions suitable for making CBGa or CBG; optionally providing an overlay; and recovering CBGa or CBG from the genetically modified host cell, the overlay, or the medium.

In another aspect, the invention provides for a genetically modified host cell capable of producing CBDa or CBD, wherein the genetically modified host cell contains one or more heterologous nucleic acids that each, independently, encodes an amino acid sequence that is at least 80% identical to the amino acid sequence of SEQ ID NOS: 12, 15, 16, or 20.

In one embodiment, the genetically modified host cell contains one or more of: a signal sequence or portion thereof; a linker or portion thereof; or a carrier protein or portion thereof. In one embodiment, the signal sequence or portion thereof comprises the amino acid sequence of SEQ ID NOS: 9, 13, 17, or 19. In one embodiment, the linker or portion thereof comprises the amino acid sequence of SEQ ID NOS: 10 or 18. In one embodiment, the carrier protein or portion thereof comprises the amino acid sequence of SEQ ID NOS: 11 or 14.

In one embodiment, the genetically modified host cell is a yeast cell or a yeast strain. In one embodiment, the yeast cell or the yeast strain is Saccharomyces cerevisiae.

In another aspect, the invention provides for a fermentation composition, wherein the fermentation composition contains a genetically modified host cell capable of producing CBDa or CBD; optionally an overlay; and CBDa or CBD produced by the genetically modified host cell. In one embodiment, the fermentation composition contains a genetically modified host cell of the present invention.

In another aspect, the invention provides for a method for producing CBDa or CBD, involving: culturing a genetically modified host cell capable of producing CBDa or CBD in a medium in the presence of CBGa or CBG and with a carbon source under conditions suitable for making CBDa or CBD; optionally providing an overlay; and recovering CBDa or CBD from the genetically modified host cell, the overlay, or the medium. In one embodiment, the genetically modified host cell capable of producing CBDa or CBD is a genetically modified host cell of the present invention.

In another aspect, the invention provides for a method for producing CBDa or CBD, involving: culturing a first genetically modified host cell capable of producing CBGa or CBG and a second genetically modified host cell capable of producing CBDa or CBD in a medium with a carbon source under conditions suitable for making CBGa, CBG, CBDa, or CBD; optionally providing an overlay; and recovering CBDa or CBD from the second genetically modified host cell, the overlay, or the medium. In one embodiment, the first genetically modified host cell is a genetically modified host cell of the present invention. In one embodiment, the second genetically modified host cell is a genetically modified host cell of the present invention.

In another aspect, the invention provides for a fermentation composition, wherein the fermentation composition contains a first genetically modified host cell capable of producing CBGa or CBG; a second genetically modified host cell capable of producing CBDa or CBD; and optionally an overlay. In one embodiment, the first genetically modified host cell is a genetically modified host cell of the present invention. In one embodiment, the second genetically modified host cell is a genetically modified host cell of the present invention. In one embodiment, the fermentation composition contains CBGa produced by the first genetically modified host cell, CBG produced by the first genetically modified host cell, CBDa produced by the second genetically modified host cell, or CBD produced by the second genetically modified host cell.

### BRIEF DESCRIPTION OF THE DRAWINGS

FIG. 1 is a schematic of the cannabinoid biosynthetic pathway.

FIG. 2 is a graph showing bioconversion of purified CBGa to CBDa by a CBDaS production strain. Purified CBGa was fed to the strain at 1 g/L on day 0, and the strain was incubated with shaking at 30 C. The ratio of CBDa to CBGa+CBDa was measured for 4 subsequent days. Error bars indicate +/−1 standard deviation.

FIG. 3 is a graph showing bioconversion of CBGa to CBDa, where CBGa was produced by a CBGa production strain (Strain ID 1) and CBDa was produced by a CBDaS production strain (Strain ID 2). Equal volumes of the Strain ID1 and Strain ID 2 were mixed incubated with shaking at 30 C. The ratio of CBDa to CBGa+CBDa was measured for 4 subsequent days. Error bars indicate +/−1 standard deviation.

FIG. 4 is a graph showing bioconversion of CBGa to CBDa, where CBGa was produced by a CBGa production strain (Strain ID 1), and CBDa was produced by one of four CBDaS production strains (Strain IDs 3-6). Equal volumes of the Strain ID 1 and Strain ID 2 were mixed incubated with shaking at 30 C. The CBDaS production strain consisted of either 1) cell pellets resuspended in fresh BSM media (with no sugar), 2) clarified broth supernatant (no cells), or 3) whole-cell broth. The ratio of CBDa to CBGa+CBDa was measured for 4 subsequent days. Error bars indicate +/−1 standard deviation.

FIG. 5 is a graph showing bioconversion of CBGa in sunflower oil (5% or 20%) to CBDa by a CBDaS production strain (Strain ID 2). Either 5% or 20% CBGa concentrate by volume was added to a CBDaS production strain 1) clarified broth supernatant (no cells), or 2) whole-cell broth. The ratio of CBDa to CBGa+CBDa was measured after 1 day and after 4 days. Error bars indicate +/−1 standard deviation.

### DETAILED DESCRIPTION OF THE INVENTION

### Definitions

As used herein the singular forms “a,” “an,” and, “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromene (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” a cannabinoid is one that contains the enzymes necessary for production of the cannabinoid according to the cannabinoid biosynthetic pathway.

As used herein, the term “exogenous” refers to a substance or compound that originated outside an organism or cell. The exogenous substance or compound can retain its normal function or activity when introduced into an organism or host cell described herein.

As used herein, the term “fermentation composition” refers to a composition which contains genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refer to a set of at least two different coding sequences, where the coding sequences encode enzymes that catalyze different parts of a synthetic pathway to form a desired product (e.g., a cannabinoid). In a genetic pathway a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of cannabinoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the term “heterologous” refers to what is not normally found in nature. The term “heterologous compound” refers to the production of a compound by a cell that does not normally produce the compound, or to the production of a compound at a level not normally produced by the cell. For example, a cannabinoid can be a heterologous compound.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “medium” refers to culture medium and/or fermentation medium.

The terms “modified,” “recombinant” and “engineered,” when used to describe a host cell described herein, refer to host cells or organisms that do not exist in nature, or express compounds, nucleic acids or proteins at levels that are not expressed by naturally occurring cells or organisms.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as CLUSTAL, BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

The term “yield” refers to production of a compound by a host cell, expressed as the amount of compound produced per amount of carbon source consumed by the host cell, by weight.

### High Efficiency Production of Cannabinoids

In some embodiments, the disclosure features a host cell capable of producing a cannabinoid, such as CBGa or CBG. In some embodiments, the host cell comprises one or more heterologous nucleic acids that each, independently, encodes an acetyl-CoA thiolase, an HMG-CoA synthase, an HMG-CoA reductase, a mevalonate kinase, a phosphomevalonate kinase, a mevalonate pyrophosphate decarboxylase, an IPP:DMAPP isomerase, an acyl-activating enzyme, a tetraketide synthase, an olivetolic acid cyclase, a cannabigerolic acid synthase (CBGaS), or a geranyl pyrophosphate synthase.

In some embodiments, the acyl-activating enzyme comprises the amino acid sequence of SEQ ID NO: 1. In some embodiments, the tetraketide synthase comprises the amino acid sequence of SEQ ID NO: 2. In some embodiments, the olivetolic acid cyclase comprises the amino acid sequence of SEQ ID NO: 3. In some embodiments, the CBGaS comprises the amino acid sequence of SEQ ID NO: 4. In some embodiments, the geranyl pyrophosphate synthase comprises the amino acid sequence of SEQ ID NO: 5.

In some embodiments, the host cell comprises one or more heterologous nucleic acids that each, independently, encodes a pyruvate decarboxylase. In some embodiments, the pyruvate decarboxylase comprises the amino acid sequence of SEQ ID NO: 6. In some embodiments, the host cell comprises modified expression of an aldehyde dehydrogenase. In some embodiments, the aldehyde dehydrogenase comprises the amino acid sequence of SEQ ID NO: 7. In some embodiments, the host cell comprises modified expression of an acetyl-coenzyme A synthetase. In some embodiments, the acetyl-coenzyme A synthetase comprises the amino acid sequence of SEQ ID NO: 8.

In some embodiments, the host cell is a yeast cell or a yeast strain. In some embodiments, the yeast cell or the yeast strain is Saccharomyces cerevisiae.

In some embodiments, the disclosure features a fermentation composition. In some embodiments, the fermentation composition comprises a host cell capable of producing CBGa or CBG, optionally an overlay (as described in, for example U.S. Patent Application No. 63/196,887), and CBGa or CBG produced by the host cell.

In some embodiments, the disclosure features a method for producing CBGa or CBG. In some embodiments, the method for producing CBGa or CBG comprises culturing a host cell capable of producing CBGa or CBG with a carbon source under conditions suitable for making CBGa or CBG, optionally providing an overlay, and recovering CBGa or CBG from the genetically modified host cell, the overlay, or the medium.

In some embodiments, the disclosure features a host cell capable of producing CBDa or CBD. In some embodiments, the host cell comprises one or more heterologous nucleic acids that each, independently, encodes a CBDaS. In some embodiments, the host cell comprises an amino acid sequence that is at least 80% identical to the amino acid sequence of SEQ ID NOS: 12, 15, 16, or 20.

In some embodiments, the host cell comprises a signal sequence or a portion thereof, a linker or a portion thereof, or a carrier protein or a portion thereof. In some embodiments, the signal sequence or portion thereof comprises the amino acid sequence of SEQ ID NOS: 9, 13, 17, or 19. In some embodiments, the linker or portion thereof comprises the amino acid sequence of SEQ ID NOS: 10 or 18. In some embodiments, the carrier protein or portion thereof comprises the amino acid sequence of SEQ ID NOS: 11 or 14.

In some embodiments, the host cell is a yeast cell or a yeast strain. In some embodiments, the yeast cell or the yeast strain is Saccharomyces cerevisiae.

In some embodiments, the disclosure features a fermentation composition. In some embodiments, the fermentation composition comprises a genetically modified host cell capable of producing CBDa or CBD, optionally an overlay, and CBDa or CBD produced by the genetically modified host cell.

In some embodiments, the disclosure features a method for producing CBDa or CBD. In some embodiments, the method for producing CBDa or CBD comprises culturing a genetically modified host cell capable of producing CBDa or CBD in a medium in the presence of CBGa and with a carbon source under conditions suitable for making CBDa or CBD, and recovering CBDa or CBD from the genetically modified host cell or the medium. In some embodiments, the CBGa is supplied in the form of an overlay. In some embodiments, the genetically modified host cell comprises an amino acid sequence that is at least 80% identical to the amino acid sequence of SEQ ID NOS: 12, 15, 16, or 20.

In some embodiments, the disclosure features a method for producing CBDa. In some embodiments, the method comprises culturing a first genetically modified host cell capable of producing CBGa or CBG and a second genetically modified host cell capable of producing CBDa or CBD in a medium with a carbon source under conditions suitable for making CBGa, CBG, CBDa, or CBD; optionally providing an overlay; and recovering CBDa or CBD from the second genetically modified host cell capable, the overlay, or the medium.

In some embodiments, the disclosure features a fermentation composition. In some embodiments, the fermentation composition comprises a first genetically modified host cell capable of producing CBGa or CBG, a second genetically modified host cell capable of producing CBDa or CBD, and optionally an overlay. In some embodiments, the fermentation composition further comprises CBGa produced by the first genetically modified host cell, CBG produced by the first genetically modified host cell, CBDa produced by the second genetically modified host cell, or CBD produced by the second genetically modified host cell.

### Cannabinoid Biosynthetic Pathway

In an aspect, a host cell described herein includes one or more nucleic acids encoding one or more enzymes of a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used by a tetraketide synthase (TKS) to produce tetraketide-CoA, which is used by an olivetolic acid cyclase (OAC) to produce olivetolic acid, which is used by a geranyl pyrophosphate (GPP) synthase and a cannabigerolic acid synthase (CBGaS) to produce a cannabigerolic acid (CBGa), which is used by a cannabidiolic acid synthase (CBDaS) to produce a cannabidiolic acid (CBDa). In some embodiments, CBGa or CBDa spontaneously decarboxylate, including upon heating, to form CBG and CBD, respectively. In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, a GPP synthase, a CBGaS, or a CBDaS. In some embodiments, the precursor, substrate, or intermediate in the cannabinoid pathway is hexanoate, olivetol, olivetolic acid, or CBGa. In some embodiments, the host cell does not contain the precursor, substrate or intermediate in an amount sufficient to produce the cannabinoid or a precursor of the cannabinoid. In some embodiments, the host cell does not contain hexanoate at a level or in an amount sufficient to produce the cannabinoid in an amount over 10 mg/L. In some embodiments, the heterologous genetic pathway encodes at least one enzyme selected from the group consisting of an AAE, a TKS, an OAC, a GPP synthase, a CBGaS, and a CBDaS. In some embodiments, the genetically modified host cell includes an AAE, TKS, OAC, a GPP synthase, a CBGaS, and a CBDaS.

The cannabinoid pathway, including the enzymes discussed in the following paragraphs, is described in U.S. Pat. No. 10,563,211, the disclosure of which is incorporated herein by reference.

In some embodiments, a host cell includes a heterologous acyl activating enzyme (AAE) such that the host cell is capable of producing a cannabinoid. The AAE may be from Cannabis sativa or may be an enzyme from another plant, fungal, or bacterial source which has been shown to have AAE activity in the cannabinoid biosynthetic pathway, resulting in the production of the cannabinoid precursor hexanoyl-CoA.

In some embodiments, a host cell includes a heterologous tetraketide synthase (TKS) such that the host cell is capable of producing a cannabinoid. A TKS uses the hexanoyl-CoA precursor to generate tetraketide-CoA. The TKS may be from Cannabis sativa or may be an enzyme from another plant, fungal, or bacterial source which has been shown to have TKS activity in the cannabinoid biosynthetic pathway, resulting in the production of the cannabinoid precursor tetraketide-CoA.

In some embodiments, a host cell includes a heterologous cannabigerolic acid synthase (CBGaS) such that the host cell is capable of producing a cannabinoid. A CBGaS uses the olivetolic acid precursor and geranyl pyrophosphate (GPP) precursor to generate cannabigerolic acid (CBGa). The CBGaS may be from Cannabis sativa or may be an enzyme from another plant, fungal, or bacterial source which has been shown to have CBGaS activity in the cannabinoid biosynthetic pathway, resulting in the production of the cannabinoid CBGa.

In some embodiments, a host cell includes a heterologous GPP synthase such that the host cell is capable of producing a cannabinoid. A GPP synthase uses the product of the isoprenoid biosynthesis pathway precursor to generate CBGa together with a prenyltransferase enzyme. The GPP synthase may be from Cannabis sativa or may be an enzyme from another plant, fungal, or bacterial source which has been shown to have GPP synthase activity in the cannabinoid biosynthetic pathway, resulting in the production of the cannabinoid CBGa.

In some embodiments, a host cell includes a heterologous CBDaS such that the host cell is capable of producing a cannabinoid. A CBDaS uses the CBGa precursor to generate CBDa. The CBDaS may be from Cannabis sativa or may be an enzyme from another plant, fungal, or bacterial source which has been shown to have CBDaS activity in the cannabinoid biosynthetic pathway, resulting in the production of the cannabinoid CBDa.

The host cell may further express other heterologous enzymes in addition to AAE, TKS, GPP synthase, CBGaS, and/or CBDaS. For example, in some embodiments, a host cell includes a heterologous olivetolic acid cyclase (OAC) such that the host cell is capable of producing a cannabinoid. An OAC uses the tetraketide-CoA precursor to generate olivetolic acid. The OAC may be from Cannabis sativa or may be an enzyme from another plant or fungal source which has been shown to have OAC activity in the cannabinoid biosynthetic pathway, resulting in the production of the cannabinoid precursor olivetolic acid. In some embodiments, the host cell may include a heterologous nucleic acid that encodes at least one enzyme from the mevalonate biosynthetic pathway. Enzymes which make up the mevalonate biosynthetic pathway may include but are not limited to an acetyl-CoA thiolase, a HMG-CoA synthase, a HMG-CoA reductase, a mevalonate kinase, a phosphomevalonate kinase, a mevalonate pyrophosphate decarboxylase, and an IPP:DMAPP isomerase. In some embodiments, the host cell includes a heterologous nucleic acid that encodes the acetyl-CoA thiolase, the HMG-CoA synthase, the HMG-CoA reductase, the mevalonate kinase, the phosphomevalonate kinase, the mevalonate pyrophosphate decarboxylase, and the IPP:DMAPP isomerase of the mevalonate biosynthesis pathway.

In some embodiments, the host cell may express heterologous enzymes of the central carbon metabolism. Enzymes of the central carbon metabolism may include an acetyl-CoA synthase, an aldehyde dehydrogenase, and a pyruvate decarboxylase. In some embodiments, the host cell includes heterologous nucleic acids that independently encode an acetyl-CoA synthase, and/or an aldehyde dehydrogenase, and/or a pyruvate decarboxylase. In some embodiments, the acetyl-CoA synthase and the aldehyde dehydrogenase from Saccharomyces cerevisiae, and the pyruvate decarboxylase from Zymomonas mobilis.

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons more frequently. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) can be considered homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used for comparing a molecule sequence to a database containing a large number of sequences from different organisms is the computer algorithm BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous kinase genes, proteins, or enzymes, techniques may include, but are not limited to, cloning a gene by PCR using primers based on a published sequence of an kinase gene/enzyme or by degenerate PCR using degenerate primers designed to amplify a conserved region among kinase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Modified Host Cells

In one aspect, provided herein are host cells comprising at least one enzyme of the cannabinoid biosynthetic pathway. In some embodiments, the cannabinoid biosynthetic pathway contains a genetic regulatory element, such as a nucleic acid sequence, that is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of a cannabinoid and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the cannabinoid biosynthetic pathway, thereby increasing production of the cannabinoid. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway contains the galactose-responsive regulatory elements described in Westfall et al. (PNAS (2012) vol. 109: E111-118). In some embodiments, the host cell lacks the gall gene and is unable to metabolize galactose, but galactose can still induce galactose-regulated genes.

TABLE A
Exemplary GAL Promoter Sequences

| Promoter | Sequence |
|---|---|
| pGAL1 | SEQ ID NO: 158 |
| pGAL10 | SEQ ID NO: 159 |
| pGAL2 | SEQ ID NO: 160 |
| pGAL3 | SEQ ID NO: 161 |
| pGAL7 | SEQ ID NO: 162 |
| pGAL4 | SEQ ID NO: 163 |

In some embodiments, the galactose regulation system used to control expression of one or more enzymes of the cannabinoid biosynthetic pathway is re-configured such that it is no longer induced by the presence of galactose. Instead, the gene of interest will be expressed unless repressors, which may be maltose in some strains, are present in the medium.

In some embodiments, the genetic regulatory element is a maltose-responsive promoter. In some embodiments, maltose negatively regulates expression of the cannabinoid biosynthetic pathway, thereby decreasing production of the cannabinoid. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Pat. No. 10,563,229, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

TABLE B
Exemplary MAL Promoter Sequences

| Promoter | Sequence |
|---|---|
| pMAL1 | SEQ ID NO: 164 |
| pMAL2 | SEQ ID NO: 165 |
| pMAL11 | SEQ ID NO: 166 |
| pMAL12 | SEQ ID NO: 167 |
| pMAL31 | SEQ ID NO: 168 |
| pMAL32 | SEQ ID NO: 169 |

In some embodiments, the heterologous genetic pathway is regulated by a combination of the maltose and galactose regulons.

In some embodiments, the recombinant host cell does not contain, or expresses a very low level of (for example, an undetectable amount), a precursor (e.g., hexanoate) required to make the cannabinoid. In some embodiments, the precursor (e.g., hexanoate) is a substrate of an enzyme in the cannabinoid biosynthetic pathway.

### Yeast Strains

In some embodiments, yeast strains useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Methods of Making the Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein. Methods for transforming host cells are described in “Laboratory Methods in Enzymology: DNA,” edited by Jon Lorsch, Volume 529, (2013); and U.S. Pat. No. 9,200,270 to Hsieh, Chung-Ming, et al., and references cited therein.

### Methods for Producing a Cannabinoid

In another aspect, methods are provided for producing a cannabinoid are described herein. In some embodiments, the method decreases expression of the cannabinoid. In some embodiments, the method includes culturing a host cell comprising at least one enzyme of the cannabinoid biosynthetic pathway described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in less than 0.001 mg/L of cannabinoid or a precursor thereof.

In some embodiments, the method is for decreasing expression of a cannabinoid or precursor thereof. In some embodiments, the method includes culturing a host cell comprising an AAE, and/or a TKS, and/or a CBGaS, and/or a GPP synthase, and/or CBDaS described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in the production of less than 0.001 mg/L of a cannabinoid or a precursor thereof.

In some embodiments, the method increases the expression of a cannabinoid. In some embodiments, the method includes culturing a host cell comprising an AAE, and/or a TKS, and/or a CBGaS, and/or a GPP synthase, and/or CBDaS described herein in a medium comprising the exogenous agent, wherein the exogenous agent increases expression of the cannabinoid. In some embodiments, the exogenous agent is galactose. In some embodiments, the method further includes culturing the host cell with the precursor or substrate required to make the cannabinoid.

In some embodiments, the method increases the expression of a cannabinoid product or precursor thereof. In some embodiments, the method includes culturing a host cell comprising a heterologous cannabinoid pathway described herein in a medium comprising an exogenous agent, wherein the exogenous agent increases the expression of the cannabinoid or a precursor thereof. In some embodiments, the exogenous agent is galactose. In some embodiments, the method further includes culturing the host cell with a precursor or substrate required to make the cannabinoid or precursor thereof. In some embodiments, the precursor required to make the cannabinoid or precursor thereof is hexanoate. In some embodiments, the combination of the exogenous agent and the precursor or substrate required to make the cannabinoid or precursor thereof produces a higher yield of cannabinoid than the exogenous agent alone.

In some embodiments, the cannabinoid or a precursor thereof is cannabidiolic acid (CBDa), cannabidiol (CBD), cannabigerolic acid (CBGa), or cannabigerol (CBG).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing cannabinoids provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cannabinoids may also occur in these culture conditions, but at undetectable levels (with detection limits being about <0.1 g/1). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may be supplemented with hexanoic acid or hexanoate as a precursor for the cannabinoid biosynthetic pathway. The hexanoic acid may have a concentration of less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid) hexanoic acid.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermenter and maintained in the range of from about 1 g/L to about 100 g/L, more preferably in the range of from about 2 g/L to about 50 g/L, and yet more preferably in the range of from about 5 g/L to about 20 g/L. Alternatively, the glucose concentration in the culture medium is maintained below detection limits. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Transformation of Heterologous Nucleic Acids into Yeast Cells

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) medium at 30° C. with shaking (200 rpm), diluted to an OD₆₀₀ of 0.1 in 100 mL YPD, and grown to an OD₆₀₀ of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000×g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that required expression of the endonuclease F-Cph1, the donor DNA included a plasmid carrying the F-CphI gene expressed under the yeast TDH3 promoter. F-CphI endonuclease expressed in such a manner cuts a specific recognition site engineered in a host strain to facilitate integration of the target gene of interest. Following a heat shock at 42° C. for 40 min, cells were recovered overnight in YPD medium before plating on selective medium. When applicable, DNA integration was confirmed by colony PCR with primers specific to the integrations.

### Example 2: Culturing of Yeast

For routine strain characterization in a 96-well-plate format, yeast colonies were picked into a 1.1-mL-per-well capacity 96-well ‘Pre-Culture plate’ filled with 360 μL per well of pre-culture medium. Pre-culture medium consisted of Bird Seed Media (BSM, originally described by van Hoek et al., Biotech. and Bioengin., 68, 2000, 517-23) at pH 5.05 with 14 g/L sucrose, 7 g/L maltose, 3.75 g/L ammonium sulfate, and 1 g/L lysine. Cells were cultured at 28° C. in a high capacity microtiter plate incubator shaking at 1000 rpm and 80% humidity for 3 days until the cultures reached carbon exhaustion.

The growth-saturated cultures were sub-cultured by taking 14.4 μL from the saturated cultures and diluting into a 2.2 mL per well capacity 96-well ‘production plate’ filled with 360 μL per well of production medium. Production medium consisted of BSM at pH 5.05 with 40 g/L sucrose, and 3.75 g/L ammonium sulfate. Cells in the production medium were cultured at 30° C. in a high capacity microtiter plate shaker at 1000 rpm and 80% humidity for an additional 3 days prior to bioconversion.

### Example 3: Analytical Methods for Product Extraction and Titer Determination

Samples were analyzed for HTAL, PDAL, olivetol, olivetolic acid, CBGa, and CBDa on a weight per volume basis, by the method below. All measurements were performed by reverse phase ultra-high pressure liquid chromatography and ultraviolet detection (UPLC-UV) using Thermo Vanquish Flex Binary UHPLC System with a Vanquish Diode Array Detector HL.

TABLE 1
Mobile Phases and Column Information

| | |
|---|---|
| Mobile Phase A: | 99.9% water + 0.1% Formic Acid, 5 mM ammonium formate |
| Mobile Phase B: | 99.9% acetonitrile + 0.1% Formic acid |
| Column | Thermo Scientific Accucore Polar Premium C18 100 mm × |
| | 2.1 mm × 2.6 um, Thermo P/N 28026-103030 |
| Guard Column | Thermo Scientific Guard Cartridge, 4 PK, P/N 28103014001 |
| Guard Column holder | Uniguard Holder, ThermoFisher, 4.0-4.6 mm, P/N 850-00 |

TABLE 2
Gradient Method

| | Flow | | | |
|---|---|---|---|---|
| Time | [mL/min] | % A | % B | Curve |
| 0.00 | 1.2 | 70 | 30 | 5 |
| 1.00 | 1.2 | 20 | 80 | 5 |
| 1.75 | 1.2 | 12.5 | 87.5 | 5 |
| 1.80 | 1.2 | 70 | 30 | 5 |
| 2.1 | 1.2 | 70 | 30 | 5 |

TABLE 3
Autosampler Parameters

| Draw speed | 2.00 | ul/s |
|---|---|---|
| Dispense speed | 5.00 | ul/s |
| Injection wash mode | Both (before and after draw) | |
| Injection wash time | 5.0 | s |
| Injection wash speed | 10.0 | ul/s |
| Sample puncture - puncture offset | 0 | um |
| Temperature control | 8 | C. |

TABLE 4
Column Compartment Settings

| Temperature control | On | |
|---|---|---|
| Temperature | 50.0 | C. |
| Ready temp delta | 0.50 | C. |
| Equilibration time | 1.0 | min |

| | |
|---|---|
| Thermostatting mode | Still air |
| Fan Speed | 5 |

TABLE 5
Detector Settings

| | | |
|---|---|---|
| UV-Vis Channel 1 Wavelength | 270 | nm |
| Data collection rate | 50.0 | Hz |
| Response time | 0.010 | s |
| Peak width | 0.100 | min |

Analytes were identified by retention time compared to an authentic standard. The peak areas were used to generate the linear calibration curve for each analyte.

At the conclusion of the incubation of the production plate, methanol was added to each well such that the final concentration was 67% (v/v) methanol. An impermeable seal was added, and the plate was shaken at 1000 rpm for 30 seconds to lyse the cells and extract cannabinoids. The plate was centrifuged for 30 seconds at 200×g to pellet cell debris. 300 μL of the clarified sample was moved to an empty 1.1-mL-capacity 96-well plate and sealed with a foil seal. The sample plate was stored at −20 C until analysis.

### Example 4: Generation of a CBGa Production Strain

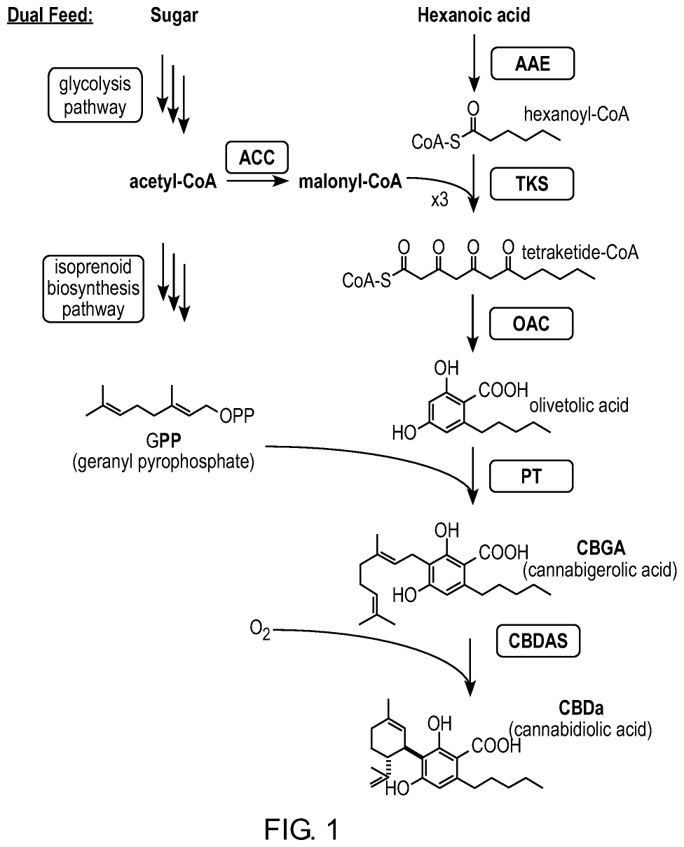

A CBGa production strain (Strain ID 1) was created from the maltose-switchable Saccharomyces cerevisiae strain by expressing the genes of the mevalonate pathway under the control of native GAL promoters. This strain contained the following chromosomally integrated mevalonate pathway genes from S. cerevisiae: acetyl-CoA thiolase (ERG10), HMG-CoA synthase (ERG13), HMG-CoA reductase (HMGR), mevalonate kinase (ERG12), phosphomevalonate kinase (ERG8), mevalonate pyrophosphate decarboxylase (MVD1), and IPP:DMAPP isomerase (IDI1). In addition, the strain contained copies of five heterologous enzymes involved in the cannabinoid biosynthetic pathway (FIG. 1): acyl-activating enzyme (AAE) (SEQ ID NO: 1), tetraketide synthase (TKS) (SEQ ID NO: 2), olivetolic acid cyclase (OAC) (SEQ ID NO: 3), and cannabigerolic acid synthase (CBGaS) (SEQ ID NO: 4) from Stachybotrys chartarum, as well as geranyl pyrophosphate synthase (GPPS) from Streptomyces aculeolatus (SEQ ID NO: 5), all under the control of GAL regulated promoters. To increase flux to cytosolic acetyl-CoA, pyruvate decarboxylase (PDC) from Zymomonas mobilis (SEQ ID NO: 6), and overexpression of S. cerevisiae aldehyde dehydrogenase (ALD6) (SEQ ID NO: 7) and acetyl-coenzyme A synthetase 1 (ACS1) (SEQ ID NO: 8) were included in the engineering. All heterologous genes described herein were codon optimized for S. cerevisiae utilizing suitable algorithms. FIG. 1 shows a schematic of the biosynthetic pathway to CBGa utilized in this strain.

### Example 5: Generation of a CBDaS Bioconversion Strain

A series of CBDaS expression strains (Strain IDs 2-6) (Table 6) were created from the maltose-switchable Saccharomyces cerevisiae strain by expressing different Cannabis sativa CBDaS expression constructs under the control of native GAL promoters (Construct IDs 1-5). The five different constructs consisted of three different yeast surface display constructs (Strain IDs 2-4), where CBDaS was fused to the N-terminus of a native yeast cell wall protein and thus covalently attached to the cell wall, and two different secretion constructs (Strain IDs 5 and 6). Strain ID 6 used the same Komagataella phaffii PEP4 signal sequence fusion as described by Zirpel et al. (2015, Biotechnol. Lett., 37(9):1869-75) for THCaS, and Zirpel et al. (2018, J. Biotechnol., 284:17-26) for CBDaS.

All strains contained a single copy of chromosomally integrated CBDaS. The strains did not express any additional heterologous genes, as CBDaS only requires molecular oxygen as a cosubstrate in addition to CBGa.

TABLE 6
Construct Table of CBDaS Production Strains

| | | | | | Carrier |
|---|---|---|---|---|---|
| | CBDaS | Signal | CBDaS | Linker | protein |
| Construct ID | Strain ID | sequence | sequence | sequence | sequence |
| Construct ID 1 | Strain ID 2 | SeqID 9 | SeqID 12 | SeqID 10 | SeqID 11 |
| Construct ID 2 | Strain ID 3 | SeqID 13 | SeqID 15 | SeqID 10 | SeqID 14 |
| Construct ID 3 | Strain ID 4 | SeqID 9 | SeqID 16 | SeqID 10 | SeqID 11 |
| Construct ID 4 | Strain ID 5 | SeqID 17 | SeqID 15 | SeqID 18 | |
| Construct ID 5 | Strain ID 6 | SeqID 19 | SeqID 20 | | |

### Example 6: Bioconversion of Purified CBGa to CBDa

CBDaS whole-cell bioconversion was performed with CBDaS Strain ID 2 using a method similar to that described in Zirpel et al. (2015, Biotechnol. Lett., 37(9):1869-75) for THCaS, and Zirpel et al. (2018, J. Biotechnol., 284:17-26) for CBDaS. Saccharomyces cerevisiae was used instead of Komagataella phaffii. CBDaS bioconversion has not been previously reported with S. cerevisiae.

CBDaS Strain ID 2 expressed CBDaS using yeast surface display, where CBDaS was fused to the N-terminus of a truncation of the native S. cerevisiae cell wall protein SAG1. A signal sequence from the native S. cerevisiae cell wall protein CWP2 was added to the N-terminus of CBDaS in addition to a KEX2 protease recognition site.

CBDaS conversion was defined as the ratio of CBDa to CBDa+CBGa, and describes the relative progress of the CBDaS reaction. A conversion ratio of 1 indicated that the reaction went to completion, and no additional CBGa was available for consumption.

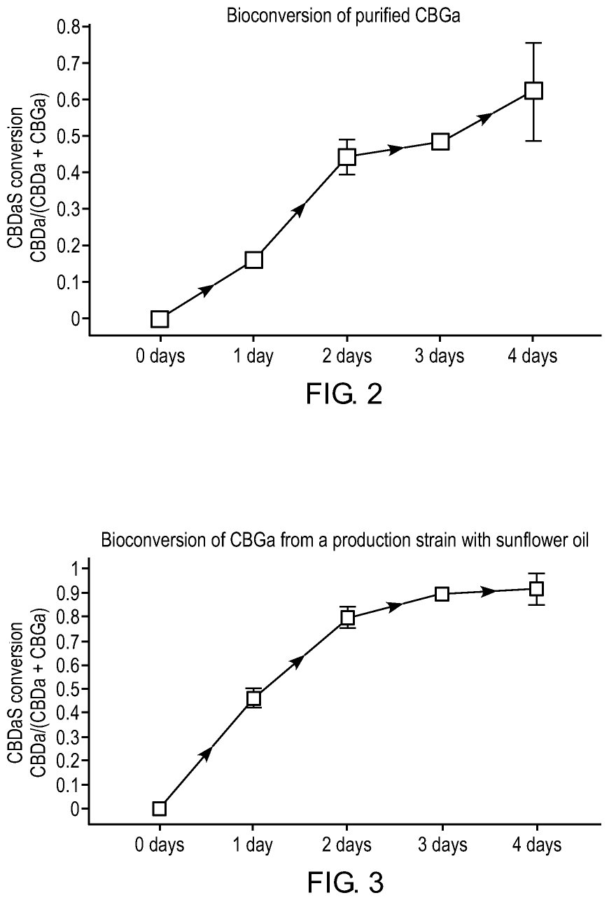

CBDaS Strain ID 2 was grown using the standard production conditions described in Example 2. Pure CBGa was dissolved in ethanol at 100 g/L, and then added directly to the CBDaS production culture to a final concentration of 1 g/L CBGa. The culture was incubated in microtiter plates at 1000 rpm at 30° C. for 0, 1, 2, 3, or 4 days before titer measurement as described in Example 3. The pure CBGa was steadily converted to CBDa over the 4-day experiment, with CBDaS conversion increasing on average by about 0.15 per day (FIG. 2) (CBD titers, although not routinely measured, were detected at low levels).

### Example 7: Bioconversion of CBGa from a Production Strain to CBDa with Overlay

CBDaS whole-cell bioconversion was again performed with CBDaS Strain ID 2, using a CBGa producer strain as the CBGa feedstock.

CBGa production Strain ID 1 was grown in a bio-reactor for 5 days at 30° C. in Bird Seed Media (BSM, originally described by van Hoek et al., Biotech. and Bioengin., 68, 2000, 517-23) at pH 5 with 15 g/L sucrose, 15 g/L maltose, 8 g/L KH₂PO₄, 6.15 g/L MgSO₄*7H₂O, 7 g/L (NH₄)₂SO₄, and 10% by weight high-oleic sunflower oil as an overlay. The tanks were fed regularly with sucrose, hexanoic acid, additional BSM, and additional high oleic sunflower oil, along with ammonium hydroxide to maintain pH 5.

CBDaS Strain ID 2 was grown using the standard production conditions described in Example 2.

CBGa was fed to the bioconversion using crude bioreactor whole-cell broth from CBGa production Strain ID 1. No purification or separation was performed on the CBGa production strain broth. Equal volumes of Strain ID 1 and Strain ID 2 were mixed in a microtiter plate after briefly vortexing and incubated at 1000 rpm and 30° C. for 0, 1, 2, 3, or 4 days before titer measurement as described in Example 3. The CBGa produced by Strain ID 1 in the bioreactor was converted to CBDa by Strain ID 2 over the 4-day experiment, approaching saturation (CBDaS conversion of 1) by day 2 (FIG. 3) (CBD titers, although not routinely measured, were detected at low levels).

### Example 8: Bioconversion Using Different CBDaS Configurations (No Overlay)

CBDaS whole-cell bioconversion was performed using four different CBDaS expression constructs, corresponding to CBDaS strain IDs 3-6.

Strain IDs 3 and 4 expressed CBDaS using yeast surface display, where CBDaS was fused to the N-terminus of a truncation of the native S. cerevisiae cell wall protein SAG1 or FLO5, respectively. In both cases, a native yeast signal sequence was fused to the N-terminus of CBDaS.

Strain IDs 5 and 6 expressed CBDaS secretion constructs consisting of a signal sequence fused to the N-terminus of CBDaS. Strain ID 6 used the same Komagataella phaffii PEP4 signal sequence fusion as described by Zirpel et al. (2015, Biotechnol. Lett., 37(9):1869-75) for THCaS, and Zirpel et al. (2018, J. Biotechnol., 284:17-26) for CBDaS.

CBDaS production Strains IDs 3-6 were grown using the standard production conditions described in Example 2. After growth, the CBDaS production strains were fractionated using centrifugation into either 1) cell pellet resuspended in fresh BSM media (with no sugar), 2) clarified broth supernatant (no cells), or 3) whole-cell broth.

CBGa production Strain ID 1 was grown in microtiter plates as described in Example 2 except the production medium contained 2 mM hexanoic acid to trigger CBGa production. No additional purification or separation was performed on the CBGa strains.

Equal volumes of CBGa strain Strain ID 1 and fraction 1, 2, or 3 of CBDaS Strain IDs 3-6 were mixed in a microtiter plate, and incubated at 1000 rpm at 30° C. for 3 days before titer measurement as described in Example 3. CBGa Strain ID 1 was resuspended by briefly vortexing, and delivered to the bioconversion as crude whole cell broth.

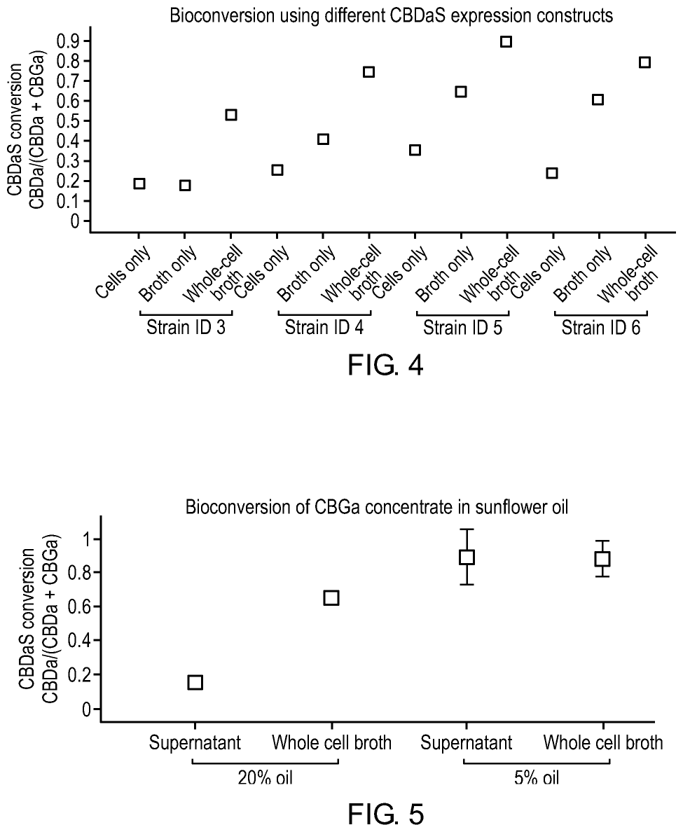

All four CBDaS constructs produced CBDa (FIG. 4) (CBD titers, although not routinely measured, were detected at low levels). In general, more activity was associated with the cell pellet (“cells only”) than with the broth only (e.g. supernatant), although the highest activity came from whole cell broth in all cases.

### Example 9: Bioconversion of CBGa Concentrate in Oil to CBDa

CBDaS whole-cell bioconversion was performed using Strain ID 2, using CBGa produced by Strain ID 1 concentrated in high-oleic sunflower oil as the CBGa feedstock.

The CBGa production Strain ID 1 was grown in a bioreactor as described in Example 7. The crude CBGa production strain broth was fractionated by centrifugation to retain the high-oleic sunflower oil overlay. The sunflower oil overlay was processed to bring the CBGa concentration to about 10 g/L.

CBDaS Strain ID 2 was grown using the standard production conditions described in Example 2. The CBDaS strain was fractionated into either 1) clarified broth supernatant (no cells), or 2) whole-cell broth.

Either 5% or 20% by volume CBGa in oil was added to the CBDaS strain in microtiter plates, and incubated at 1000 rpm and 30° C. for 4 days before titer measurement as described in Example 3. All four conditions led to CBDa production (FIG. 5), with the lower dose of CBGa concentrate (5%) reaching saturation for both the supernatant and whole cell broth fractions of the CBDaS strain (CBD titers, although not routinely measured, were detected at low levels). CBDaS strain whole cell broth produced substantially more CBDa than CBDaS strain supernatant.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

All publications, patents, and patent applications cited in this specification are herein incorporated by reference as if each individual publication or patent application were specifically and individually indicated to be incorporated by reference.

SEQUENCE APPENDIX

SEQ ID NO: 1-Acyl-activating enzyme from Cannabis sativa
MGKNYKSLDSVVASDFIALGITSEVAETLHGRLAEIVCNYGAATPQTWINIANHILSPDL
PFSLHQMLFYGCYKDFGPAPPAWIPDPEKVKSTNLGALLEKRGKEFLGVKYKDPISSFSH
FQEFSVRNPEVYWRTVLMDEMKISFSKDPECILRRDDINNPGGSEWLPGGYLNSAKNCL
NVNSNKKLNDTMIVWRDEGNDDLPLNKLTLDQLRKRVWLVGYALEEMGLEKGCAIAI
DMPMHVDAVVIYLAIVLAGYVVVSIADSFSAPEISTRLRLSKAKAIFTQDHIIRGKKRIPL
YSRVVEAKSPMAIVIPCSGSNIGAELRDGDISWDYFLERAKEFKNCEFTAREQPVDAYTN
ILFSSGTTGEPKAIPWTQATPLKAAADGWSHLDIRKGDVIVWPTNLGWMMGPWLVYAS
LLNGASIALYNGSPLVSGFAKFVQDAKVTMLGVVPSIVRSWKSTNCVSGYDWSTIRCFS
SSGEASNVDEYLWLMGRANYKPVIEMCGGTEIGGAFSAGSFLQAQSLSSFSSQCMGCTL
YILDKNGYPMPKNKPGIGELALGPVMFGASKTLLNGNHHDVYFKGMPTLNGEVLRRHG
DIFELTSNGYYHAHGRADDTMNIGGIKISSIEIERVCNEVDDRVFETTAIGVPPLGGGPEQ
LVIFFVLKDSNDTTIDLNQLRLSFNLGLQKKLNPLFKVTRVVPLSSLPRTATNKIMRRVL
RQQFSHFE
SEQ ID NO: 2-Tetraketide synthase from Cannabis sativa
MNHLRAEGPASVLAIGTANPENILLQDEFPDYYFRVTKSEHMTQLKEKFRKICDKSMIR
KRNCFLNEEHLKQNPRLVEHEMQTLDARQDMLVVEVPKLGKDACAKAIKEWGQPKSK
ITHLIFTSASTTDMPGADYHCAKLLGLSPSVKRVMMYQLGCYGGGTVLRIAKDIAENNK
GARVLAVCCDIMACLFRGPSESDLELLVGQAIFGDGAAAVIVGAEPDESVGERPIFELVS
TGQTILPNSEGTIGGHIREAGLIFDLHKDVPMLISNNIEKCLIEAFTPIGISDWNSIFWITHP
GGKAILDKVEEKLHLKSDKFVDSRHVLSEHGNMSSSTVLFVMDELRKRSLEEGKSTTGD
GFEWGVLFGFGPGLTVERVVVRSVPIKY
SEQ ID NO: 3-Olivetolic acid cyclase from Cannabis sativa
MAVKHLIVLKFKDEITEAQKEEFFKTYVNLVNIIPAMKDVYWGKDVTQKNKEEGYTHI
VEVTFESVETIQDYIIHPAHVGFGDVYRSFWEKLLIFDYTPRK
SEQ ID NO: 4-CBGaS from Stachybotrys chartarum
MSAKVSPMAYTNPRYETGPLSLIPKPIVPYFELMRFELPHGYYLGYFPHLVGIMYGASA
GPERLPARDLVFQALLYVGWTFAMRGAGCAWNDNIDQDFDRKTERCRTRPIARGAVST
TAGHVFAVAGVALAFLCLSPLPTECHQLGVLFTVLSVIYPFCKRFTNFAQVILGMTLAA
NFILAAYGAGLPALEQPYTRPTMSATLAITLLVVFYDVVYARQDTADDLKSGVKGMAV
LFRNHIEVLLAVLTCTIGGLLAATGVSVGNGPYYFLFSVAGLTVALLAMIGGIRYRIFHT
WNGYSGWFYVLAIINLMSGYFIEYLDNAPILARGS
SEQ ID NO: 5-Geranyl pyrophosphate synthase from Streptomyces aculeolatus
MTTEVTSFTGAGPHPAASVRRITDDLLQRVEDKLASFLTAERDRYAAMDERALAAVDA
LTDLVTSGGKRVRPTFCITGYLAAGGDAGDPGIVAAAAGLEMLHVSALIHDDILDNSAQ
RRGKPTIHTLYGDLHDSHGWRGESRRFGEGIGILIGNLALVYSQELVCQAPPAVLAEWH
RLCSEVNIGQCLDVCAAAEFSADPELSRLVALIKSGRYTIHRPLVMGANAASRPDLAAA
YVEYGEAVGEAFQLRDDLLDAFGDSTETGKPTGLDFTQHKMTLLLGWAMQRDTHIRTL
MTEPGHTPEEVRRRLEDTEVPKDVERHIADLVEQGRAAIADAPIDPQWRQELADMA VR
AAYRTN
SEQ ID NO: 6-Pyruvate decarboxylase from Zymomonas mobilis
MSYTVGTYLAERLVQIGLKHHFAVAGDYNLVLLDNLLLNKNMEQVYCCNELNCGFSA
EGYARAKGAAAAVVTYSVGALSAFDAIGGAYAENLPVILISGAPNNNDHAAGHVLHHA
LGKTDYHYQLEMAKNITAAAEAIYTPEEAPAKIDHVIKTALREKKPVYLEIACNIASMPC
AAPGPASALFNDEASDEASLNAAVEETLKFIANRDKVAVLVGSKLRAAGAEEAAVKFA
DALGGAVATMAAAKSFFPEENPHYIGTSWGEVSYPGVEKTMKEADAVIALAPVENDYS
TTGWTDIPDPKKLVLAEPRSVVVNGIRFPSVHLKDYLTRLAQKVSKKTGALDFFKSLNA
GELKKAAPADPSAPLVNAEIARQVEALLTPNTTVIAETGDSWFNAQRMKLPNGARVEY
EMQWGHIGWSVPAAFGYAVGAPERRNILMVGDGSFQLTAQEVAQMVRLKLPVIIFLIN
NYGYTIEVMIHDGPYNNIKNWDYAGLMEVENGNGGYDSGAGKGLKAKTGGELAEAIK
VALANTDGPTLIECFIGREDCTEELVKWGKRVAAANSRKPVNKLL
SEQ ID NO: 7-Aldehyde dehydrogenase 6 from Saccharomyces cerevisiae
MTKLHFDTAEPVKITLPNGLTYEQPTGLFINNKFMKAQDGKTYPVEDPSTENTVCEVSS
ATTEDVEYAIECADRAFHDTEWATQDPRERGRLLSKLADELESQIDLVSSIEALDNGKTL
ALARGDVTIAINCLRDAAAYADKVNGRTINTGDGYMNFTTLEPIGVCGQIIPWNFPIMM
LAWKIAPALAMGNVCILKPAAVTPLNALYFASLCKKVGIPAGVVNIVPGPGRTVGAALT
NDPRIRKLAFTGSTEVGKSVAVDSSESNLKKITLELGGKSAHLVFDDANIKKTLPNLVNG
IFKNAGQICSSGSRIYVQEGIYDELLAAFKAYLETEIKVGNPFDKANFQGAITNRQQFDTI
MNYIDIGKKEGAKILTGGEKVGDKGYFIRPTVFYDVNEDMRIVKEEIFGPVVTVAKFKTL
EEGVEMANSSEFGLGSGIETESLSTGLKVAKMLKAGTVWINTYNDFDSRVPFGGVKQSG
YGREMGEEVYHAYTEVKAVRIKL
SEQ ID NO: 8-Acetyl-coenzyme A synthetase 1 from Saccharomyces cerevisiae
MSPSAVQSSKLEEQSSEIDKLKAKMSQSASTAQQKKEHEYEHLTSVKIVPQRPISDRLQP
AIATHYSPHLDGLQDYQRLHKESIEDPAKFFGSKATQFLNWSKPFDKVFIPDSKTGRPSF
QNNAWFLNGQLNACYNCVDRHALKTPNKKAIIFEGDEPGQGYSITYKELLEEVCQVAQ
VLTYSMGVRKGDTVAVYMPMVPEAIITLLAISRIGAIHSVVFAGFSSNSLRDRINDGDSK
VVITTDESNRGGKVIETKRIVDDALRETPGVRHVLVYRKTNNPSVAFHAPRDLDWATEK
KKYKTYYPCTPVDSEDPLFLLYTSGSTGAPKGVQHSTAGYLLGALLTMRYTFDTHQED
VFFTAGDIGWITGHTYVVYGPLLYGCATLVFEGTPAYPNYSRYWDIIDEHKVTQFYVAP
TALRLLKRAGDSYIENHSLKSLRCLGSVGEPIAAEVWEWYSEKIGKNEIPIVDTYWQTES
GSHLVTPLAGGVTPMKPGSASFPFFGIDAVVLDPNTGEELNTSHAEGVLAVKAAWPSFA
RTIWKNHDRYLDTYLNPYPGYYFTGDGAAKDKDGYIWILGRVDDVVNVSGHRLSTAEI
EAAIIEDPIVAECAVVGENDDLTGQAVAAFVVLKNKSNWSTATDDELQDIKKHLVFTVR
KDIGPFAAPKLIILVDDLPKTRSGKIMRRILRKILAGESDQLGDVSTLSNPGIVRHLIDSVKL
SEQ ID NO: 9-Signal sequence from Saccharomyces cerevisiae
MQFSTVASVAFVALANFVAARR
SEQ ID NO: 10-Linker
AEAAAKEAAAKA
SEQ ID NO: 11-Carrier protein from Saccharomyces cerevisiae
ASAKSSFISTTTTDLTSINTSAYSTGSISTVETGNRTTSEVISHVVTTSTKLSPTATTSLTIA
QTSIYSTDSNITVGTDIHTTSEVISDVETISRETASTVVAAPTSTTGWTGAMNTYISQFTSS
SFATINSTPIISSSAVFETSDASIVNVHTENITNTAAVPSEEPTFVNATRNSLNSFCSSKQPS
SPSSYTSSPLVSSLSVSKTLLSTSFTPSVPTSNTYIKTKNTGYFEHTALTTSSVGLNSFSETA
VSSQGTKIDTFLVSSLIAYPSSASGSQLSGIQQNFTSTSLMISTYEGKASIFFSAELGSIIFLL
LSYLLF
SEQ ID NO: 12-CBDaS from Cannabis sativa
MKCSTFSFWFVCKIIFFFFSFNIQTSIANPTENFLKCFSQYIDNNATNDKLVYTQDDPLYM
SVLNSTIHNDRESSDTTPKPLVIVTPSHVSHIQGTILCSKKVGLQIRTRSGGHDSEGMSYIS
QDPFVIVDLRNMRSIKIDVHSQTAWVEAGATLGEVYYNVNEKNENLTLAAGYCPTVCA
GGHFGGGGYGPLMRSYGLAADNIIDAHLVNVDGKVLDRKSMGEDLFWALRGGGAESF
GIIVAWKIRLVAVPKSTMFSVKKIMEIHELVKLVNKWQNIAYKYDKDLLLMTHFITRNIT
DNQGNNKTAIHTYFSCVFLGGVDSLVDLMNKTFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLCKVKTLVDPNNFFRNEQSIP
PLPRHRH
SEQ ID NO: 13-Signal sequence from Saccharomyces cerevisiae
MQLLRCFSIFSVIASVLA
SEQ ID NO: 14-Carrier protein from Saccharomyces cerevisiae
FYPSNGTSVISSSVISSSVTSSLVTSSSFISSSVISSSTTTSTSIFSESSTSSVIPTSSSTSGSSES
KTSSASSSSSSSSISSESPKSPTNSSSSLPPVTSATTGQETASSLPPATTTKTSEQTTLVTVTS
CESHVCTESISSAIVSTATVTVSGVTTEYTTWCPISTTETTKQTKGTTEQTKGTTEQTTET
TKQTTVVTISSCESDICSKTASPAIVSTSTATINGVTTEYTTWCPISTTESKQQTTLVTVTS
CESGVCSETTSPAIVSTATATVNDVVTVYPTWRPQTTNEQSVSSKMNSATSETTTNTGA
AETKTAVTSSLSRFNHAETQTASATDVIGHNNSVVSVSETGNTKSLTSSGLSTMSQQPRS
TPASSMVGYSTASLEISTYAGSANSLLAGSGLSVFIASLLLAII
SEQ ID NO: 15-CBDaS from Cannabis sativa
MKCSTFSFWFVCKIIFFFFSFNIQTSIANPRENFLKCFSQYIPNNATNLKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNESLSLAAGYCPTVCA
GGHFGGGGYGPLMRSYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKSFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIP
PLPRHRH
SEQ ID NO: 16-CBDaS from Cannabis sativa
MKCSTFSFWFVCKIIFFFFSFNIQTSIANPRENFLKCFSQYIPNNATNLKLVYTQNNPLYM
SVLNSTIHNLRFSSDTTPKPLVIVTPSHVSHIQGTILCSKKVGLQIRTRSGGHDSEGMSYIS
QVPFVIVDLRNMRSIKIDVHSQTAWVEAGATLGEVYYWVNEKNENLTLAAGYCPTVCA
GGHFGGGGYGPLMRSYGLAADNIIDAHLVNVHGKVLDRKSMGEDLFWALRGGGAESF
GIIVAWKIRLVAVPKSTMFSVKKIMEIHELVKLVNKWQNIAYKYDKDLLLMTHFITRNIT
DNQGKNKTAIHTYFSSVFLGGVDSLVDLMNKTFPELGIKKTDCRQLSWIDTIIFYSGVVN
YDTDNFNKEILLDRSAGQNGAFKIKLDYVKKPIPESVFVQILEKLYEEDIGAGMYALYPY
GGIMDEISESAIPFPHRAGILYELWYICSWEKQEDNEKHLNWIRNIYNFMTPYVSQNPRL
AYLNYRDLDIGINDPKNPNNYTQARIWGEKYFGKNFDRLVKVKTLVDPNNFFRNEQSIP
PLPRHRH
SEQ ID NO: 17-Signal sequence from Saccharomyces cerevisiae
MRQVWFSWIVGLFLCFFNVSSARR
SEQ ID NO: 18-Linker
EPEPEPEPEPEPEPEASAKALLSQPLLLI
SEQ ID NO: 19-Signal sequence from Komagataella pastoris
MIFDGTTMSIAIGLLSTLGIGAEA
SEQ ID NO: 20-CBDaS from Cannabis sativa
NPRENFLKCFSQYIPNNATNLKLVYTQNNPLYMSVLNSTIHNLRFSSDTTPKPLVIVTPSH
VSHIQGTILCSKKVGLQIRTRSGGHDSEGMSYISQVPFVIVDLRNMRSIKIDVHSQTAWV
EAGATLGEVYYWVNEKNENLTLAAGYCPTVCAGGHFGGGGYGPLMRSYGLAADNIID
AHLVNVHGKVLDRKSMGEDLFWALRGGGAESFGIIVAWKIRLVAVPKSTMFSVKKIME
IHELVKLVNKWQNIAYKYDKDLLLMTHFITRNITDNQGKNKTAIHTYFSSVFLGGVDSL
VDLMNKTFPELGIKKTDCRQLSWIDTIIFYSGVVNYDTDNFNKEILLDRSAGQNGAFKIK
LDYVKKPIPESVFVQILEKLYEEDIGAGMYALYPYGGIMDEISESAIPFPHRAGILYELWY
ICSWEKQEDNEKHLNWIRNIYNFMTPYVSQNPRLAYLNYRDLDIGINDPKNPNNYTQAR
IWGEKYFGKNFDRLVKVKTLVDPNNFFRNEQSIPPLPRHRH

1. A genetically modified host cell capable of producing CBGa or CBG, wherein the genetically modified host cell comprises one or more heterologous nucleic acids that each, independently, encodes:
a) an acetyl-CoA thiolase;
b) an HMG-CoA synthase;
c) an HMG-CoA reductase;
d) a mevalonate kinase;
e) a phosphomevalonate kinase;
f) a mevalonate pyrophosphate decarboxylase;
g) an IPP:DMAPP isomerase;
h) an acyl-activating enzyme comprising the amino acid sequence of SEQ ID NO: 1;
i) a tetraketide synthase comprising the amino acid sequence of SEQ ID NO: 2;
j) an olivetolic acid cyclase comprising the amino acid sequence of SEQ ID NO: 3;
k) a cannabigerolic acid synthase (CBGaS) comprising the amino acid sequence of SEQ ID NO: 4; and
l) a geranyl pyrophosphate synthase comprising the amino acid sequence of SEQ ID NO: 5.

2. The genetically modified host cell of claim 1, further comprising one or more of:
a) one or more heterologous nucleic acids that each, independently, encodes a pyruvate decarboxylase comprising the amino acid sequence of SEQ ID NO: 6;
b) modified expression of an aldehyde dehydrogenase comprising the amino acid sequence of SEQ ID NO: 7; or
c) modified expression of an acetyl-coenzyme A synthetase comprising the amino acid sequence of SEQ ID NO: 8.

3. The genetically modified host cell of claim 1, wherein the genetically modified host cell is a yeast cell or a yeast strain.

4. The genetically modified host cell of claim 3, wherein the yeast cell or the yeast strain is Saccharomyces cerevisiae.

5. A fermentation composition comprising:
a) the genetically modified host cell of claim 1;
b) optionally an overlay; and
c) CBGa or CBG produced by the genetically modified host cell.

6. A method for producing CBGa or CBG, comprising:
a) culturing the genetically modified host cell of claim 1 in a medium with a carbon source under conditions suitable for making CBGa or CBG;
b) optionally providing an overlay; and
c) recovering CBGa or CBG from the genetically modified host cell, the overlay, or the medium.

7. A genetically modified host cell capable of producing CBDa or CBD, wherein the genetically modified host cell comprises one or more heterologous nucleic acids that each, independently, encodes an amino acid sequence that is at least 80% identical to the amino acid sequence of SEQ ID NOS: 12, 15, 16, or 20.

8. The genetically modified host cell of claim 7, further comprising one or more of:
a. a signal sequence or portion thereof;
b. a linker or portion thereof, or
c. a carrier protein or portion thereof.

9. The genetically modified host cell of claim 8, wherein:
a) the signal sequence or portion thereof comprises the amino acid sequence of SEQ ID NOS: 9, 13, 17, or 19;
b) the linker or portion thereof comprises the amino acid sequence of SEQ ID NOS: 10 or 18; or
c) the carrier protein or portion thereof comprises the amino acid sequence of SEQ ID NOS: 11 or 14.

10. The genetically modified host cell of claim 7, wherein the genetically modified host cell is a yeast cell or a yeast strain.

11. The genetically modified host cell of claim 10, wherein the yeast cell or the yeast strain is Saccharomyces cerevisiae.

12. A fermentation composition comprising:
a) a genetically modified host cell of claim 7;
b) optionally an overlay; and
c) CBDa or CBD produced by the genetically modified host cell.

13. The fermentation composition of claim 12, wherein the genetically modified host cell is the genetically modified host cell of claim 7.

14. A method for producing CBDa or CBD, comprising:
a) culturing a genetically modified host cell capable of producing CBDa or CBD in a medium in the presence of CBGa or CBG and with a carbon source under conditions suitable for making CBDa or CBD;
b) optionally providing an overlay; and
c) recovering CBDa or CBD from the genetically modified host cell, the overlay, or the medium.

15. The method of claim 14, wherein the genetically modified host cell capable of producing CBDa or CBD is the genetically modified host cell of claim 7.

16. The method of claim 14, wherein the CBGa or CBG is present in an overlay.

17. A method for producing CBDa or CBD comprising:
a) culturing a first genetically modified host cell capable of producing CBGa or CBG and a second genetically modified host cell capable of producing CBDa or CBD in a medium with a carbon source under conditions suitable for making CBGa, CBG, CBDa, or CBD;
b) optionally providing an overlay; and
c) recovering CBDa or CBD from the second genetically modified host cell, the overlay, or the medium.

18. (canceled)

19. A fermentation composition comprising:
a) a first genetically modified host cell capable of producing CBGa or CBG;
b) a second genetically modified host cell capable of producing CBDa or CBD; and
c) optionally an overlay.

20. (canceled)

21. (canceled)

22. The method of claim 14, wherein the medium comprises whole-cell broth or clarified broth supernatant of a genetically modified host cell capable of producing CBGa or CBG.

23. The method of claim 17, wherein the second genetically modified host cell comprises a cell pellet or whole-cell broth of the second genetically modified host cell.


SEQUENCE LISTING (20 sequences)

SEQ ID NO: 1 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 2 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 3 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 4 - protein, 329 aa, Stachybotrys chartarum
MSAKVSPMAYTNPRYETGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLFTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 5 - protein, 357 aa, Streptomyces aculeolatus
MTTEVTSFTGAGPHPAASVRRITDDLLQRVEDKLASFLTAERDRYAAMDERALAAVDALTDLVTSGGKRVRPTFCITGYLAAGGDAGDPGIVAAAAGLEMLHVSALIHDDILDNSAQRRGKPTIHTLYGDLHDSHGWRGESRRFGEGIGILIGNLALVYSQELVCQAPPAVLAEWHRLCSEVNIGQCLDVCAAAEFSADPELSRLVALIKSGRYTIHRPLVMGANAASRPDLAAAYVEYGEAVGEAFQLRDDLLDAFGDSTETGKPTGLDFTQHKMTLLLGWAMQRDTHIRTLMTEPGHTPEEVRRRLEDTEVPKDVERHIADLVEQGRAAIADAPIDPQWRQELADMAVRAAYRTN

SEQ ID NO: 6 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 7 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 8 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDSKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSNWSTATDDELQDIKKHLVFTVRKDIGPFAAPKLIILVDDLPKTRSGKIMRRILRKILAGESDQLGDVSTLSNPGIVRHLIDSVKL

SEQ ID NO: 9 - protein, 22 aa, Saccharomyces cerevisiae
MQFSTVASVAFVALANFVAARR

SEQ ID NO: 10 - protein, 12 aa, synthetic construct
AEAAAKEAAAKA

SEQ ID NO: 11 - protein, 321 aa, Saccharomyces cerevisiae
ASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 12 - protein, 544 aa, Cannabis sativa
MKCSTFSFWFVCKIIFFFFSFNIQTSIANPTENFLKCFSQYIDNNATNDKLVYTQDDPLYMSVLNSTIHNDRFSSDTTPKPLVIVTPSHVSHIQGTILCSKKVGLQIRTRSGGHDSEGMSYISQDPFVIVDLRNMRSIKIDVHSQTAWVEAGATLGEVYYNVNEKNENLTLAAGYCPTVCAGGHFGGGGYGPLMRSYGLAADNIIDAHLVNVDGKVLDRKSMGEDLFWALRGGGAESFGIIVAWKIRLVAVPKSTMFSVKKIMEIHELVKLVNKWQNIAYKYDKDLLLMTHFITRNITDNQGNNKTAIHTYFSCVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLCKVKTLVDPNNFFRNEQSIPPLPRHRH

SEQ ID NO: 13 - protein, 18 aa, Saccharomyces cerevisiae
MQLLRCFSIFSVIASVLA

SEQ ID NO: 14 - protein, 417 aa, Saccharomyces cerevisiae
FYPSNGTSVISSSVISSSVTSSLVTSSSFISSSVISSSTTTSTSIFSESSTSSVIPTSSSTSGSSESKTSSASSSSSSSSISSESPKSPTNSSSSLPPVTSATTGQETASSLPPATTTKTSEQTTLVTVTSCESHVCTESISSAIVSTATVTVSGVTTEYTTWCPISTTETTKQTKGTTEQTKGTTEQTTETTKQTTVVTISSCESDICSKTASPAIVSTSTATINGVTTEYTTWCPISTTESKQQTTLVTVTSCESGVCSETTSPAIVSTATATVNDVVTVYPTWRPQTTNEQSVSSKMNSATSETTTNTGAAETKTAVTSSLSRFNHAETQTASATDVIGHNNSVVSVSETGNTKSLTSSGLSTMSQQPRSTPASSMVGYSTASLEISTYAGSANSLLAGSGLSVFIASLLLAII

SEQ ID NO: 15 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 16 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLTLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 17 - protein, 24 aa, Saccharomyces cerevisiae
MRQVWFSWIVGLFLCFFNVSSARR

SEQ ID NO: 18 - protein, 29 aa, synthetic construct
EPEPEPEPEPEPEPEASAKALLSQPLLLI

SEQ ID NO: 19 - protein, 24 aa, Komagataella pastoris
MIFDGTTMSIAIGLLSTLGIGAEA

SEQ ID NO: 20 - protein, 516 aa, Cannabis sativa
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLTLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKTFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH